(19) United States
(12) Patent Application Publication
(10) Pub. No.: US 2025/0195158 A1
(43) Pub. Date: Jun. 19, 2025

(54) SURGICAL ROBOTIC SYSTEM FOR CONDUCTING A PLURALITY OF CONCURRENT COLONOSCOPIES

(71) Applicant: Covidien LP, Mansfield, MA (US)

(72) Inventors: Meir Rosenberg, Newton, MA (US); Richard O. Kuenzler, Framingham, MA (US); Brock Kopp, Colorado Springs, CO (US)

(21) Appl. No.: 18/875,852

(22) PCT Filed: Jun. 15, 2023
(86) PCT No.: PCT/US2023/025377

§ 371 Date: Dec. 17, 2024

Related U.S. Application Data
(60) Provisional application No. 63/442,513, filed on Feb. 1, 2023.
(60) Provisional application No. 63/356,528, filed on Jun. 29, 2022.

(51) Int. Cl. A61B 34/30; A61B 1/00; A61B 1/31

(52) U.S. Cl. CPC A61B 34/30; A61B 1/000094; A61B 1/31; A61B 2034/301

(57) ABSTRACT

A surgical robotic system for performing multiple simultaneous colonoscopies includes a surgical console, a control tower, and a plurality of autonomous robotic colonoscope carts. Each autonomous robotic colonoscope cart includes a robotic arm, an endoscope operably coupled to the robotic arm, and a computing device. The computing device is configured to control autonomous advancement or retraction of the endoscope through the colon and transmit images captured by the endoscope to the control tower for display on the surgical console. The computing device is also configured to analyze the images captured by the endoscope as the endoscope is advanced or retracted through the colon, tag one or more images analyzed as corresponding to an area of interest within the colon, and cause the surgical console to display the tagged image while the endoscope is within a vicinity of the area of interest within the colon.

### CROSS-REFERENCE TO RELATED APPLICATIONS

This application claims the benefit of the filing date of provisional U.S. Patent Application No. 63/356,528 filed on Jun. 29, 2022, and provisional U.S. Patent Application No. 63/442,513 filed on Feb. 1, 2023. The entire contents of the foregoing applications are incorporated by reference herein.

### FIELD

The present disclosure generally relates to a surgical robotic system for performing multiple concurrent colonoscopies. More particularly, the present disclosure is directed to a system including one or more surgical consoles for controlling a plurality of autonomous robotic colonoscope carts, each of which is conducting a colonoscopy on a different patient.

### BACKGROUND

Colorectal cancer is the third leading cause of cancer-related deaths in the United States. Prior to reaching a cancerous stage, colorectal cancer often begins in the form of small polyps, adenomas, or abnormal growths of the colon surface that, when found early, may be easily and safely removed. The preferred screening technique for polyp detection and removal is optical colonoscopy, during which an endoscope with an attached camera is inserted and guided through the colon to examine the colon wall to find polyps for removal. Since optical colonoscopy requires the attentiveness of medical professionals for identifying polyps, poor diligence or navigational skills for maneuvering the camera leads to missing identification of polyps and anomalies.

Conventional colonoscopy has been used for screening of colorectal cancer. However, as the colon is a tortuous and pliable tube with many internal ridges and sharp bends, advancement of the colonoscope is difficult and often complicated by excessive looping and stretching of the colon. This may cause significant pain and discomfort as well as a substantial risk of over-distension or even perforation of the colon. A high degree of technical expertise from the medical practitioner is therefore required for skillful manipulation of the colonoscope.

Robotics technology may be incorporated into endoscopes and would provide a number of advantages for various applications, including colonoscopy. For example, by exploiting soft deformable structures that are capable of moving effectively through a complex environment like the inside of the colon, one can significantly reduce pain and patient discomfort while minimizing the risk of colonic perforation. However, the guidance of such robotic endoscopes may still require significant technical expertise by the medical practitioner.

### SUMMARY

In accordance with aspects of the disclosure, a surgical robotic system for performing multiple simultaneous colonoscopies includes a surgical console, a control tower, and a plurality of autonomous robotic colonoscope carts. Each autonomous robotic colonoscope cart includes a robotic arm, an endoscope operably coupled to the robotic arm, and a computing device. The endoscope is configured to capture images of a colon. The computing device is configured to control autonomous advancement or retraction of the endoscope through the colon and transmit images captured by the endoscope to the control tower for display on the surgical console. The computing device is also configured to analyze the images captured by the endoscope as the endoscope is advanced or retracted through the colon, tag one or more images analyzed as corresponding to an area of interest within the colon, and cause the surgical console to display the tagged image while the endoscope is within a vicinity of the area of interest within the colon in response to an image being tagged. In an aspect, the surgical console or the control tower is configured to switch one autonomous robotic colonoscope cart of the plurality of autonomous robotic colonoscope carts from autonomous control to manual control.

In an aspect, the endoscope may include at least one of a surgical instrument for treating the colon or a working channel for receipt of a surgical instrument therethrough.

In an aspect, the computing device may be configured to determine whether an image includes surface mucosa and may cause the autonomous robotic colonoscope cart to wash the surface mucosa when it is determined that the image includes surface mucosa.

In an aspect, the tagged image may be tagged with data including a location of the area of interest in the colon and a classification of a polyp within the tagged image.

In an aspect, the computing device may be configured to cause the surgical console to display images preceding and following the area of interest within the colon for review.

In an aspect, the computing device may stop the autonomous advancement or retraction of the endoscope within the colon when an image is tagged for a preconfigured period of time or until intervention is received.

In an aspect, the surgical console may include a handle controller configured to override the autonomous advancement or retraction of the endoscope and control movement of the endoscope through the colon.

In an aspect, the computing device may be configured to detect a collapse in the colon wall and may generate an alert when the collapse is detected

In an aspect, the surgical console, the control tower, or the plurality of autonomous robotic colonoscope carts may be configured to determine which images from which autonomous robotic colonoscope cart is displayed on the surgical console.

In an aspect, surgical robotic system may include a plurality of surgical consoles.

According to another aspect of the disclosure, a surgical robotic system for performing multiple simultaneous colonoscopies is provided. The surgical robotic system includes a surgical console including a computing device. The computing device of the surgical console is configured to receive image data from a plurality of autonomous robotic colonoscope carts, each autonomous robotic colonoscope cart including an endoscope capturing a set of colonoscopy images of a respective colon, analyze the received image data to tag one or more images analyzed as corresponding to an area of interest within the colon, display each set of colonoscopy images on a display, and highlight one set of colonoscopy images displayed based on the analyzing.

In an aspect, the surgical console may be configured to highlight one set of colonoscopy images by displaying a border around the one set of colonoscopy images.

In an aspect, the surgical console may be configured to highlight one set of colonoscopy images by displaying the one set of colonoscopy images in a larger size relative to the other sets of colonoscopy images displayed.

In an aspect, the surgical console may be configured to display a tagged image and images preceding and following the area of interest while the endoscope is within a vicinity of the area of interest within the colon.

In an aspect, the surgical console may include a handle controller configured to control movement of the endoscope through the colon.

In an aspect, the surgical console may be configured to detect a collapse in a colon wall and generate an alert when the collapse is detected.

In an aspect, the system further includes a plurality of autonomous robotic colonoscope carts.

In an aspect, surgical robotic system may include a plurality of surgical consoles.

According to another aspect of the disclosure a computer-implemented method for performing multiple simultaneous colonoscopies is provided. The method includes receiving image data from a plurality of autonomous robotic colonoscope carts, where each autonomous robotic colonoscope cart includes an endoscope capturing a set of colonoscopy images of a respective colon, analyzing the received image data to tag one or more images analyzed as corresponding to an area of interest within the colon, selecting one set of colonoscopy images for display on a surgical console based on the analyzing or highlighting one set of colonoscopy images displayed on a surgical console, and switching one autonomous robotic colonoscope cart of the plurality of autonomous robotic colonoscope carts from autonomous control to manual control based on the selecting.

In an aspect, the method further includes detecting a collapse in a colon wall based on the received image data and generating an alert when the collapse is detected. Additionally, or alternatively, the selecting one set of colonoscopy images for display on a surgical console may be based on the analyzing and the detecting the collapse in the colon wall.

In an aspect, the method further includes determining whether an image includes surface mucosa and causing the autonomous robotic colonoscope cart corresponding to the image to wash the surface mucosa when it is determined that the image includes surface mucosa.

According to another aspect of the disclosure, a surgical robotic system for performing an autonomous colonoscopy procedure is provided. The surgical robotic system includes an autonomous robotic colonoscope cart and a surgical console operably coupled to the autonomous robotic colonoscope cart. The autonomous robotic colonoscope cart includes a robotic arm and an endoscope operably coupled to the robotic arm and configured to capture images of a colon. The surgical console is configured to receive images captured by the endoscope in real time, display the received images captured by the endoscope, receive a destination of a location input by a clinician, generate a pathway for movement of the endoscope to the received destination, and cause the autonomous robotic colonoscope cart to advance or retract the endoscope through the colon along the generated pathway to the received destination. The surgical console is further configured to analyze the images captured by the endoscope as the endoscope is advanced or retracted through the colon along the generated pathway to the received destination, and tag an image as corresponding to an area of interest within the colon based on the analysis of the images.

In an aspect, the surgical console is further configured to confirm a location of the endoscope along the pathway based on a comparison between a previously captured image of the colon and a newly captured image of the colon. The newly captured image of the colon may be captured while the endoscope is advanced or retracted along the generated pathway.

In an aspect, the destination of the location is based on a selection of a previously captured image.

In an aspect, the destination of the location is based on a selection of a point along a diagram of the colon.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

Various aspects of the present disclosure are described herein with reference to the drawings wherein:

FIG. 1A is a schematic illustration of a surgical robotic system including a control tower, a console, and a plurality of autonomous robotic colonoscope carts according to the present disclosure;

FIG. 1B is a schematic illustration of the surgical robotic system of FIG. 1A with three autonomous robotic colonoscope carts, each of which is assigned to a patient bed;

FIG. 2 is a perspective view of a surgical robotic arm of an autonomous robotic colonoscope cart of FIGS. 1A-1B according to the present disclosure;

FIG. 3 is a perspective view of an autonomous robotic colonoscope cart of the surgical robotic system of FIGS. 1A-1B according to the present disclosure;

FIG. 4 is a schematic diagram of a computer architecture of the surgical robotic system of FIGS. 1A-1B according to the present disclosure;

FIG. 5 is a flowchart of a computer-implemented method for performing a plurality of concurrent colonoscopies according to the present disclosure; and

FIG. 6 is a flowchart of a computer-implemented method for performing an autonomous colonoscopy navigation procedure.

### DETAILED DESCRIPTION

Embodiments of the presently disclosed surgical robotic systems are described in detail with reference to the drawings, in which like reference numerals designate identical or corresponding elements in each of the several views.

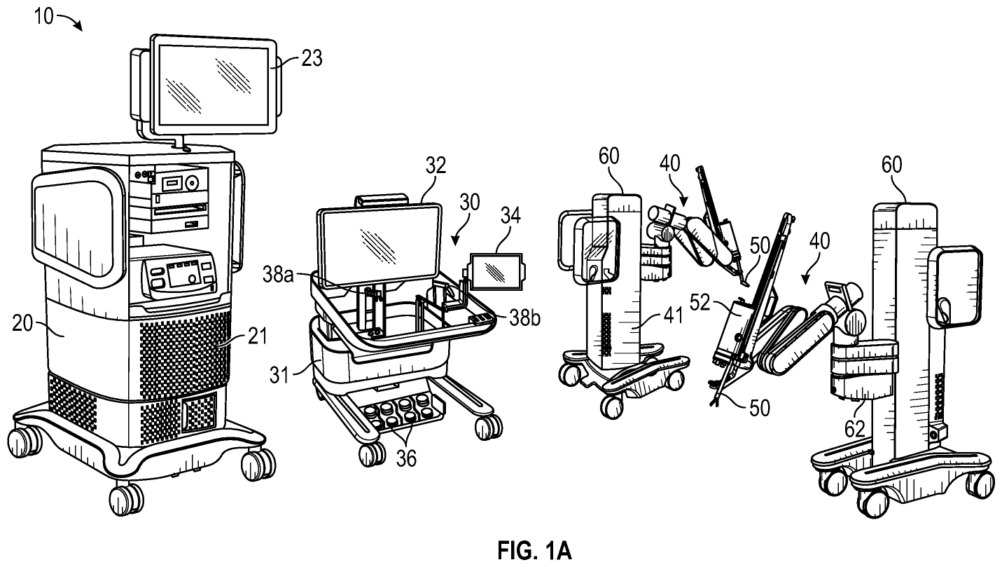

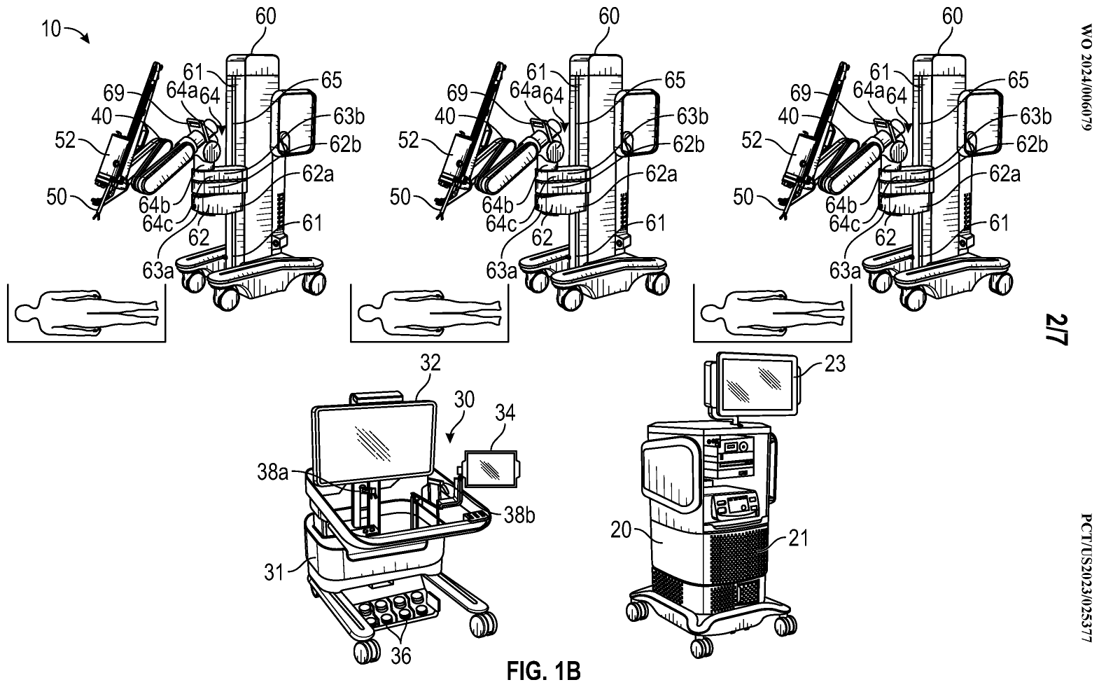

With reference to FIGS. 1A-1B, a surgical robotic system 10 includes a control tower 20, which is connected to all of the components of the surgical robotic system 10, including a surgical console 30 and a plurality of autonomous robotic colonoscope carts 60. Although FIG. 1A illustrates two autonomous robotic colonoscope carts 60 and FIG. 1B illustrates three autonomous robotic colonoscope carts 60, surgical robotic system 10 may include any number of autonomous robotic colonoscope carts 60. Each autonomous robotic colonoscope cart 60 includes one or more robotic arms 40 with an endoscope 50 (e.g., a colonoscope) operably coupled thereto. Additionally, or alternatively, the endoscope 50 may include one or more working channels configured to receive a surgical instrument therethrough for introduction into the colon during the colonoscopy, for example, to treat areas of the colon or to obtain biopsy samples.

The surgical console 30 includes a first display device 32, which displays the colonoscopy video and images generated by the endoscope 50 disposed on the robotic arms 40, and a second display device 34, which displays a user interface for controlling the surgical robotic system 10. The surgical console 30 also includes a plurality of user interface devices, such as foot pedals 36 and a pair of handle controllers 38a and 38b, which are used by a clinician to remotely control robotic arms 40 of autonomous robotic colonoscope carts 60. It is appreciated that multiple surgical consoles 30 may be incorporated into system 10 for controlling (and receiving data from) one or more autonomous robotic colonoscope carts 60.

The control tower 20 acts as an interface between the surgical console 30 and each of the plurality of autonomous robotic colonoscope carts 60. In particular, the control tower 20 is configured to control the robotic arms 40, such as to move the robotic arms 40 and the corresponding endoscope 50, based on a set of programmable instructions and/or input commands from the surgical console 30, in such a way that robotic arms 40 and the endoscope 50 execute a desired movement sequence in response to input from the foot pedals 36 and the handle controllers 38a and 38b. The control tower 20 includes a display 23 for displaying various information pertaining to the surgical robotic system 10.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 802.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

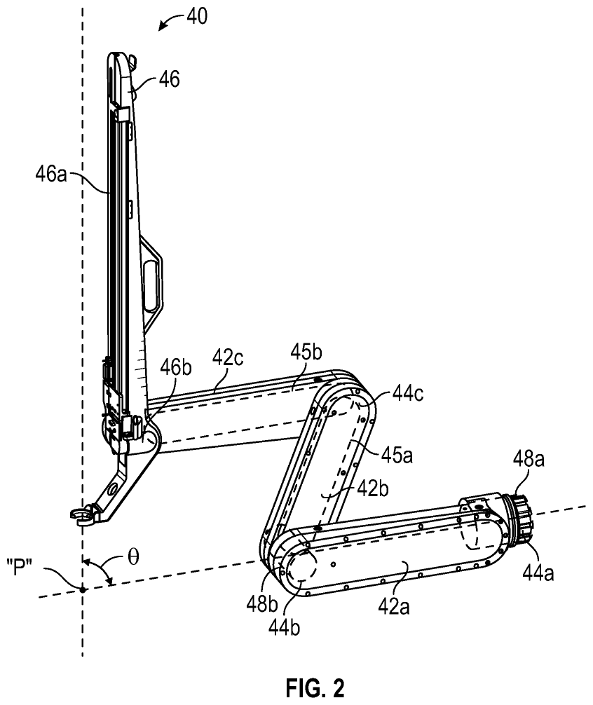

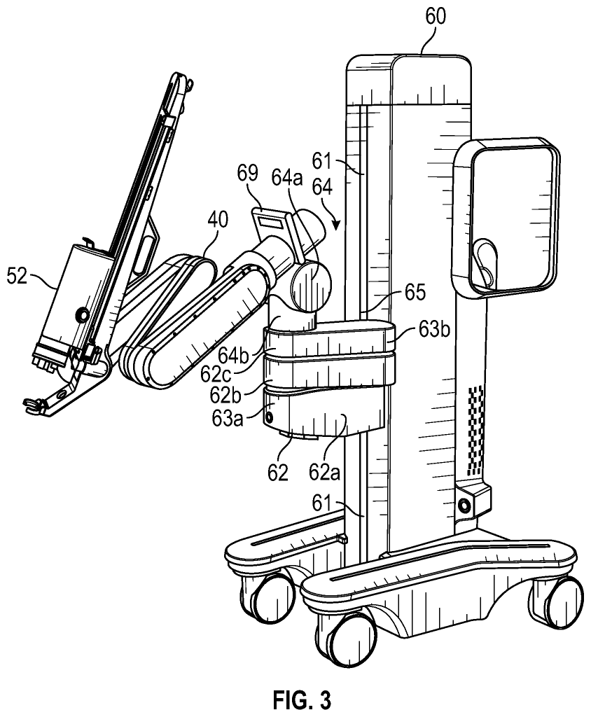

With reference to FIG. 2, each of the robotic arms 40 may include of a plurality of links 42a, 42b, 42c, which are interconnected at rotational joints 44a, 44b, 44c, respectively. The joint 44a is configured to secure the robotic arm 40 to the robotic colonoscope 60 and defines a first longitudinal axis. With reference to FIG. 3, the autonomous robotic colonoscope cart 60 includes a lift 61 and a setup arm 62, which provides a base for mounting of the robotic arm 40. The lift 61 allows for vertical movement of the setup arm 62. The autonomous robotic colonoscope cart 60 includes the cart display 69 for displaying information pertaining to the robotic arm 40 or other components of the autonomous robotic colonoscope cart 60.

The setup arm 62 includes a first link 62a, a second link 62b, and a third link 62c, which provide for lateral maneuverability of the robotic arm 40. The links 62a, 62b, 62c are interconnected at rotational joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis, which is perpendicular to a plane defined by the third link 62c, and the second actuator 64b is rotatable about a second stationary arm axis which is transverse to the first stationary arm axis. The first and second actuators 64a and 64b allow for full three-dimensional orientation of the robotic arm 40.

With reference to FIG. 2, the robotic arm 40 also includes a holder 46 defining a second longitudinal axis and configured to receive an instrument drive unit 52 (FIG. 1A) of the surgical endoscope 50 and/or the endoscope 50, which is configured to couple to an actuation mechanism of the surgical endoscope 50 and/or the endoscope 50. Instrument drive unit 52 transfers actuation forces from its actuators to the surgical endoscope 50 and/or the endoscope 50 to actuate components (e.g., end effectors) of the surgical endoscope 50 and/or components (e.g., image sensors, catheter bodies, etc.) of the endoscope 50. The holder 46 includes a sliding mechanism 46a, which is configured to move the instrument drive unit 52 along the second longitudinal axis defined by the holder 46. The holder 46 also includes a rotational joint 46b, which rotates the holder 46 relative to the link 42c.

The joints 44a and 44b include an electrical actuator 48a and 48b configured to drive the joints 44a, 44b, 44c relative to each other through a series of belts 45a and 45b or other mechanical linkages such as a drive rod, a cable, or a lever and the like. In particular, the actuator 48b of the joint 44b is coupled to the joint 44c via the belt 45a, and the joint 44c is in turn, coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. Thus, the actuator 48b controls the pitch angle θ between the first and second axes allowing for orientation of the surgical endoscope 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an electrical actuator to obviate the need for mechanical linkages.

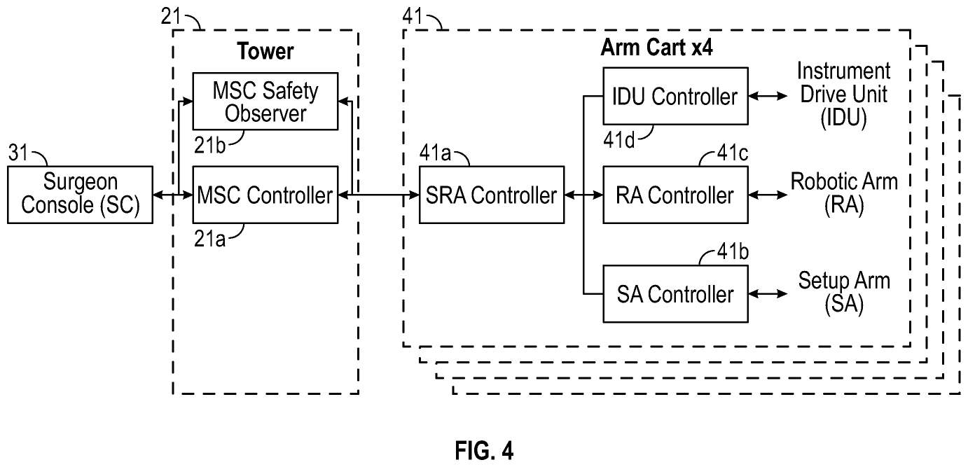

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the instrument drive unit 52 and communicates these to the computer 41 of the robotic arm 40. The controller 21a also receives back the actual joint angles and uses this information to determine force feedback commands that are transmitted back to the computer 31 of the surgical console 30 to provide haptic feedback through the handle controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the autonomous robotic colonoscope cart 60, the robotic arm 40, and the instrument drive unit 52. The main cart controller 41a also communicates actual joint angles back to the controller 21a.

The setup arm controller 41b controls each of rotational joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed-loop position control. The robotic arm controller 41c calculates a movement command based on the calculated torque. The calculated motor commands are then communicated to one or more of the electrical actuators 48a and 48b in the robotic arm 40. The actual joint positions are then transmitted by the electrical actuators 48a and 48b back to the robotic arm controller 41c.

The IDU controller 41d receives desired joint angles for the surgical endoscope 50 and/or the endoscope 50, such as wrist and jaw angles, and computes desired currents for the motors in the instrument drive unit 52. The IDU controller 41d calculates actual angles based on the motor positions and transmits the actual angles back to the main controller 41a.

The robotic arm controller 41c is configured to estimate torque imparted on the rotational joints 44a and 44b by the rigid link structure of the robotic arm 40, namely, the links 42a, 42b, 42c. Each of the rotational joints 44a and 44b houses electrical actuator 48a and 48b. High torque may be used to move the robotic arm 40 due to the heavy weight of the robotic arm 40. However, the torque may need to be adjusted to prevent damage or injury. This is particularly useful for limiting torque during collisions of the robotic arm 40 with external objects, such as other robotic arms, patient, staff, operating room (OR) equipment, etc.

Each autonomous robotic colonoscope cart 60 is configured to capture images of one patient's colon and transmit those images to one or both of the control tower 20 or the surgical console 30. The surgical console 30 may be operated by a single clinician viewing one or more colonoscopy images received from one or more of the autonomous robotic colonoscope carts 60. Each autonomous robotic colonoscope cart 60 is configured to control advancement or retraction of the respective endoscope 50 through the respective patient's colon undergoing the imaging. The control of the advancement and retraction of the endoscope 50 may be entirely autonomous, entirely manually operated by the clinician via surgical console 30, or may be a hybrid of autonomous control by the autonomous robotic colonoscope cart 60 and manual control by the clinician operating the surgical console 30. In one specific example, the clinician manually advances the endoscope 50 from the entry point to the cecum and the autonomous robotic colonoscope cart 60 automatically retracts the endoscope 50 from the cecum back to the entry point. As the clinician advances the endoscope 50, the clinician may tag images of portions of the patient's colon and upon autonomous retraction of the endoscope 50 by the autonomous robotic colonoscope cart 60, the autonomous robotic colonoscope cart 60 may perform a redundancy check of the clinician's tagged selections.

As described above, each autonomous robotic colonoscope cart 60 includes a computer 41. Although the functions described below are described as being carried out by the computer 41 of the autonomous robotic colonoscope cart 60, it is appreciated that any other component or combination of components of system 10, such as computer 21 of control tower 20 and/or computer 31 of surgical console 30, may perform some or all of the functions identified below.

The computer 41 is configured to control autonomous advancement or retraction of the endoscope 50 through the colon and transmit images captured by the endoscope 50 to the surgical console 30 either directly or through the control tower 20. Additionally, the computer 41 is configured to analyze the images captured by the endoscope 50 as the endoscope 50 is advanced or retracted through the colon and tag one or more images analyzed as corresponding to an area of interest within the colon based on the image analysis. For example, the computer 41 may perform an image analysis of each of the images to determine if an abnormality is present within the image. When an abnormality is detected, the computer 41 tags the image with data such as a location of the area of interest in the colon and a classification of a polyp within the tagged image.

Simultaneous with the tagging, or following the tagging, the computer 41 causes the surgical console 30 to display the tagged image while the endoscope 50 is within the vicinity of the area of interest, which may include minimizing the view of images from another colonoscopy being displayed on the surgical console 30. This action is effectively causing a selection of which image set, that is from which autonomous robotic colonoscope cart 60 of the plurality of autonomous robotic colonoscope carts 60 is to be displayed, or in what form it will be displayed, on the surgical console 30 for review by the clinician. As cach autonomous robotic colonoscope cart 60 is constantly analyzing its respective colonoscopy images, the user interface displayed on the surgical console 30 is constantly changing to bring the most recent and/or most urgent matters concerning one or more of the simultaneous colonoscopies to the clinician's attention.

In addition to causing the surgical console 30 to display the tagged image. depending on the severity of the cause of the tag, the computer 41 may additionally display images preceding and/or following the tagged image to provide the clinician with additional visual context of the area of interest. Further, depending on the severity of the cause of the tag, the computer 41 may also generate an alarm or other notification to alert the clinician of an alarming condition. For example, in one aspect, the computer 41 may detect a collapse in the colon wall, via image analysis of the images captured by the endoscope 50 or via other sensing means associated with the autonomous robotic colonoscope cart 60, and generate an alert when the collapse is detected. In aspects, a pressure change from an insufflator is monitored to provide a delta or rate of change of a CO2 flow rate as a compensator to detect an amount of a leak. Monitoring the amount of time/volume of gas to reach a steady state should be proportional to the volume introduced into the abdomen. A warning or other notification can be generated to notify the physician or technician to check for abdominal distension or excessive pressure to confirm a detected collapse.

In aspects, when an image is tagged, the computer 41 causes the autonomous robotic colonoscope cart 60 to stop advancing or retracting endoscope 50 through the colon for a predetermined period of time, or until a clinician intervenes via the surgical console 30.

In one aspect, the computer 41 is configured to analyze the images captured by the endoscope 50 and determine whether surface mucosa is present in the image, and if so, cause a surgical endoscope 50 of the autonomous robotic colonoscope cart 60 to wash the surface mucosa. Washing the suspicious location can be performed and repeated to better resolve and capture the surface patterns of the polyp to more accurately differentiate the lesion.

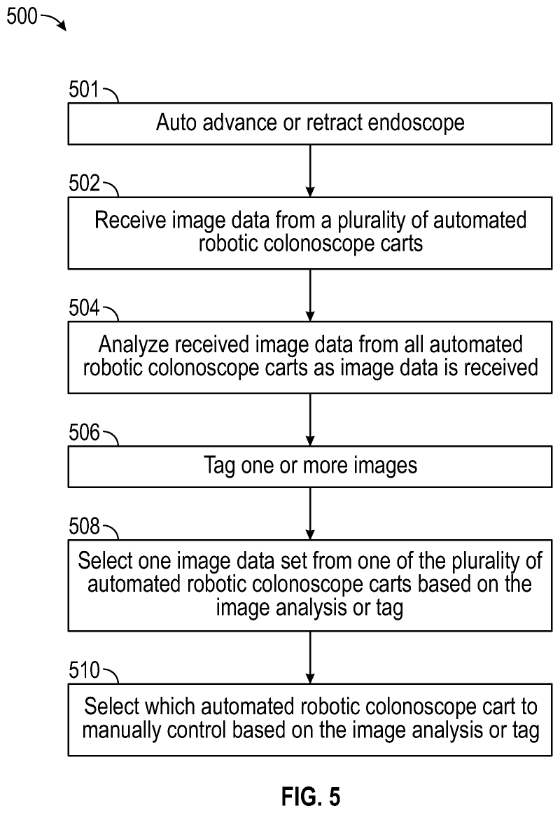

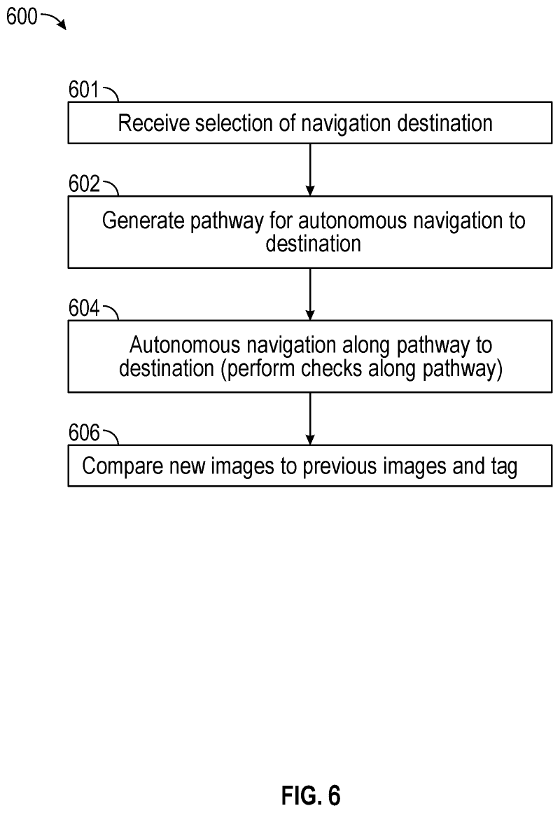

The flow diagrams of FIGS. 5-6 described below include various blocks described in an ordered sequence. However, those skilled in the art will appreciate that one or more blocks of the flow diagrams may be performed in a different order, repeated, and/or omitted without departing from the scope of the disclosure. The below description of the flow diagrams refers to various actions or tasks performed by control tower 20 of system 10, but those skilled in the art will appreciate that the control tower 20 is exemplary, and some or all of the steps may be carried out by one or more other components of system 10, such as surgical console 30 and/or autonomous robotic colonoscope cart 60, individually or in combination with each other. In various aspects, the disclosed operations can be performed by another component, device, or system. In various aspects, at least some of the operations can be implemented by firmware, programmable logic devices, and/or hardware circuitry. Other implementations are contemplated to be within the scope of the disclosure.

Initially, at step 501 of method 500, each autonomous robotic colonoscope cart 60 automatically advances its endoscope 50 through the corresponding patient's colon to capture images of the patient's colon. As described above, in aspects, the clinician may perform the initial advancement of the endoscope 50 through the patient's colon from the entry point to the cecum. This may be done manually, or via the controls of the surgical console 30 by the clinician. Thus, step 501 may begin after the clinician has completed this initial advancement of the endoscope 50, and may instead include each autonomous robotic colonoscope cart 60 automatically retracting the endoscope 50 from the cecum to the entry point. The autonomous robotic colonoscope cart 60 may record the navigation data of the endoscope 50 as the endoscope 50 is navigated from the entry point to the cecum by the clinician, and then utilize that recorded data for retracting the endoscope 50 in step 501.

In step 502, the control tower 20 receives image data from each autonomous robotic colonoscope cart 60 conducting a colonoscopy of the plurality of autonomous robotic colonoscope carts 60 conducting simultaneous colonoscopies on different patients. The image data received may be partially or fully analyzed (e.g., image processing, artificial intelligence augmentation, etc.) by the individual autonomous robotic colonoscope carts 60 transmitting the image data. Alternatively, in step 504, the control tower 20 analyzes the received image data from all of the plurality of autonomous robotic colonoscope carts 60, for example as the image data is received.

In step 506, based on the analysis in step 504, one or more of the images received are tagged by the control tower 20 as including a portion of the image of the patient's colon as of potential concern for example, as including a polyp, discoloration, or other abnormality that should be reviewed by a clinician. Depending on the reason for the tag and/or the severity of the cause for the tag, the control tower 20 may generate an alarm or other notification to alert the clinician of the tagged image. Also, the control tower 20 selects one image data set from the plurality of image data sets received from each autonomous robotic colonoscope cart 60 of the plurality of autonomous robotic colonoscope carts 60 to be displayed on the surgical console 30. In addition to selecting which image data set to display on the surgical console 30, step 508 may additionally include selecting how the image data set is presented or otherwise displayed to the clinician, for example, in relation to the other image data sets received from the other autonomous robotic colonoscope carts 60. For example, when the surgical console 30 is displaying multiple colonoscopy images, the surgical console 30 can highlight one set of the displayed images by displaying a border around the selected set of images to draw the user's attention to that set. Additionally, or alternatively, the surgical console 30 may highlight one set of colonoscopy images by displaying the one set of colonoscopy images in a larger size relative to the other sets of colonoscopy images displayed. Additionally, or alternatively, the surgical console 30 can display a tagged image and images preceding and following the area of interest while the endoscope is within a vicinity of the area of interest within the colon. This selection in step 508, ensures that a single clinician operating a single surgical console 30 can perform, or otherwise monitor, a plurality of simultaneously occurring colonoscopy procedures on a plurality of patients.

In an aspect, in step 510 the control of the specific autonomous robotic colonoscope cart 60 that captured the tagged image in step 506 is switched from autonomous control (e.g., controlled by the cach autonomous robotic colonoscope cart 60) to manual control by the clinician via the surgical console 30. Specifically, once an image is tagged in step 506, and then displayed on the surgical console 30 in step 508, the system 10 may enable manual control of the autonomous robotic colonoscope cart 60 for manual control by a clinician operating surgical console 30.

In one aspect, the clinician may initially introduce the endoscope 50 through a patient's colon, while tagging images of areas of interest during the advancement. Upon reaching the patient's cecum, the autonomous robotic colonoscope cart 60 may retract the endoscope 50 and conduct its own independent image analysis of the images of the colon captured during the retraction to either confirm the clinician's manual tags or disregard them.

With reference to FIG. 6, a method for autonomous navigation of the endoscope 50 is illustrated and described as method 600. Method 600 may be implemented: 1) before a colonoscopy procedure (e.g., to initiate an autonomous navigation to a destination within a colon); 2) during a colonoscopy procedure to initiate an autonomous navigation to a destination within the patient's colon that has not yet been imaged; 3) during the colonoscopy procedure to return to a destination within the patient's colon for reimaging; or 4) subsequent to a first colonoscopy procedure to revisit portions of the patient's colon for reimaging in a second colonoscopy procedure.

Initially, at step 601, the surgical console 30 receives a user input of a selection of a navigation destination within a patient's colon. In an aspect, the surgical console 30 displays visualization tools, for example past images of the patient's colon, a diagram or 3D rendering of the patient's colon, a location bar corresponding to a length of the patient's colon, etc. and the clinician selects the visualization tools corresponding to a desired navigation destination. A clinician may select an image of the patient's colon captured in a previous colonoscopy procedure or an image of a patient's colon captured in an in-progress colonoscopy procedure, for which the clinician desires reimaging or reassessment of those portions of the patient's colon. In aspects, revisiting an area of the patient's colon for reanalysis may be desired and beneficial where an image of a portion of the patient's colon was initially determined to be of low potential concern, but images of portions of the patient's colon captured downstream of the patient's colon have the potential to ultimately change the diagnosis based on the earlier captured upstream images. In such cases, a new analysis of the initial images or new images of that portion of the patient's colon may be performed based on the previous discoveries and observations downstream of the patient's colon.

In particular, step 601 may be implemented during a colonoscopy procedure to return the endoscope 50 to reimage a portion of the patient's colon that was previously imaged to obtain new images and/or to review live video of that portion of the colon. In addition, during the colonoscopy procedure, step 601 may be implemented to autonomously “fast-forward” the navigation of the endoscope 50 to a specific portion of the colon. In aspects, the images are tagged or associated with specific locations or coordinates such that, when a user selects an image of the patient's colon to revisit, the surgical console 30 utilizes the coordinates for the endoscope 50 associated with the selected image and assigns the coordinates as the navigation destination. Similarly, where a user selects a location along a displayed colon diagram or rendering, each point along the colon diagram or rendering may be associated with coordinates for the endoscope 50 which are assigned as the coordinates for the navigation destination.

At step 602, the surgical console 30 generates a navigation pathway from the current location of the endoscope 50 (or entry point for a new colonoscopy procedure) to the destination location based on the coordinates associated with the selection received at step 601. In aspects, the navigation pathway may be displayed on the surgical console 30 as a prompt for review and approval by the clinician and method 600 does not proceed to step 604 until the generated navigation pathway is approved by the clinician. At step 604, the surgical console 30 instructs the autonomous robotic colonoscope 60 to navigate the endoscope 50 along the generated pathway to the navigation destination based on the coordinates associated with the user selection.

During autonomous navigation of the endoscope 50, the surgical console 30 or the autonomous robotic colonoscope 60 may compare previously recorded images of the patient's colon to the real time images captured by the endoscope 50 during navigation of the endoscope 50 along the navigation pathway to: 1) confirm the accuracy of the coordinates of the endoscope 50 along the navigation path (step 604); and/or 2) determine whether any new images captured along the navigation pathway require tagging for clinician review (step 606). If a deviation between the previously captured images and the images captured during autonomous navigation of the endoscope 50 along the navigation path is detected (e.g., where previously captured images and newly captured images do not match above a predetermined threshold), then a prompt may be displayed to notify the clinician that the assumed location of the endoscope 50 is inaccurate and a reregistration of the location of the endoscope 50 may be required. Additionally or alternatively, if a deviation is detected, such as where previously captured images and newly captured images do not match above a predetermined threshold, or if a new condition is detected in a newly captured image, then that new image may be tagged indicating that a portion of the new image of the patient's colon is of potential concern e.g., as including a polyp, discoloration, or other abnormality, that should be reviewed by a clinician. The image matching and deviation detection may be accomplished by the implementation of any suitable image matching algorithm. For example, a Brute-Force Matcher, a Fast Library for Approximate Nearest Neighbors (FLANN) Matcher, or a pixel-based Means Squared Error approach may be implemented to perform the image matching and deviation detection. In one aspect, the image matching algorithm may utilize a pixel-by-pixel analysis or a feature matching technique which establishes correspondences between two images of the same scene/object and subsequently established preliminary features matches between the two images. Depending on the reason for the tag and/or the severity of the cause for the tag, the control tower 20 may generate an alarm or other notification to alert the clinician of the tagged image.

The system 10 described above aids in advancing patients quickly through the colonoscopy process and triages to the appropriate level of intervention and specialist treatment. The system 10 also enables diagnosis and treatment of patients that are the treatable and referral of the patients requiring advanced treatment. Additionally, the system 10 enables management of the appropriate polyps and identification of polyps not requiring removal. Because the system 10 tags images and associates each tagged image with a corresponding location in the colon, the system 10 facilitates repeat colonoscopies by showing the progression of any areas of interest tagged in previous colonoscopies. The location of the tagged image can be stored for return upon command (e.g., autonomous return of the endoscope to the location or digital return to the associated image.)

The system 10 may facilitate artificial intelligence (AI) to analyze the images and tag polyps and areas of interest with data including the location of the polyp, the classification of the polyp, tortuosity of the endoscope 50 at the time of the image capture, and the scene surrounding the polyp.

In aspects, the autonomous robotic colonoscope cart 60 automatically retracts the endoscope 50 through the colon based on one or more of looping amounts and positions, scenes near polyps, polyp morphology, or other factors which may or may not have been obtained during advancement of the endoscope 50 through the colon.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A surgical robotic system for performing multiple simultaneous colonoscopies, the system comprising:
a surgical console;
a control tower in communication with the surgical console; and
a plurality of autonomous robotic colonoscope carts in communication with the control tower and the surgical console, each autonomous robotic colonoscope cart including:
a robotic arm;
an endoscope operably coupled to the robotic arm and configured to capture images of a colon; and
a computing device configured to:
control autonomous advancement or retraction of the endoscope through the colon;
transmit images captured by the endoscope to the control tower for display on the surgical console;
analyze the images captured by the endoscope as the endoscope is advanced or retracted through the colon;
tag an image analyzed as corresponding to an area of interest within the colon; and
cause the surgical console to display the tagged image while the endoscope is within a vicinity of the area of interest within the colon in response
to an image being tagged,
wherein at least one of the surgical console or the control tower is configured to switch one autonomous robotic colonoscope cart of the plurality of autonomous robotic colonoscope carts from autonomous control to manual control.

2. The system of claim 1, wherein the endoscope includes at least one of a surgical instrument for treating the colon or a working channel for receipt of a surgical instrument therethrough.

3. The system of claim 1, wherein the computing device is configured to determine whether an image includes surface mucosa and cause the autonomous robotic colonoscope cart to wash the surface mucosa when it is determined that the image includes surface mucosa.

4. The system of claim 1, wherein the tagged image is tagged with data including a location of the area of interest in the colon and a classification of a polyp within the tagged image.

5. The system of claim 1, wherein the computing device is configured to cause the surgical console to display images preceding and following the area of interest within the colon for review when an image is tagged.

6. The system of claim 1, wherein the computing device stops the autonomous advancement or retraction of the endoscope within the colon when an image is tagged for a preconfigured period of time or until intervention is received.

7. The system of claim 1, wherein the surgical console includes a handle controller configured to override the autonomous advancement or retraction of the endoscope and control movement of the endoscope through the colon.

8. The system of claim 1, wherein the computing device is configured to detect a collapse in a colon wall and generate an alert when the collapse is detected.

9. The system of claim 1, wherein at least one of the surgical console, the control tower, or the plurality of autonomous robotic colonoscope carts is configured to determine which images from which autonomous robotic colonoscope cart is displayed on the surgical console.

10. A surgical robotic system for performing multiple simultaneous colonoscopies, the system comprising:
a surgical console including a computing device configured to:
receive image data from a plurality of autonomous robotic colonoscope carts, each autonomous robotic colonoscope cart including an endoscope capturing a set of colonoscopy images of a respective colon;
analyze the received image data to tag one or more images analyzed as corresponding to an area of interest within the colon;
display each set of colonoscopy images on a display; and
highlight one set of colonoscopy images displayed based on the analyzing.

11. The system of claim 10, wherein the surgical console is configured to highlight one set of colonoscopy images by displaying a border around the one set of colonoscopy images.

12. The system of claim 10, wherein the surgical console is configured to highlight one set of colonoscopy images by displaying the one set of colonoscopy images in a larger size relative to the other sets of colonoscopy images displayed.

13. The system of claim 10, wherein the surgical console is configured to display a tagged image and images preceding and following the area of interest while the endoscope is within a vicinity of the area of interest within the colon.

14. The system of claim 10, wherein the surgical console includes a handle controller configured to control movement of the endoscope through the colon.

15. The system of claim 10, wherein the surgical console is configured to detect a collapse in a colon wall and generate an alert when the collapse is detected.

16. The system of claim 10, further comprising a plurality of autonomous robotic colonoscope carts.

17. A computer-implemented method for performing multiple simultaneous colonoscopies comprising:
receiving image data from a plurality of autonomous robotic colonoscope carts, each autonomous robotic colonoscope cart including an endoscope capturing a set of colonoscopy images of a respective colon;
analyzing the received image data to tag one or more images analyzed as corresponding to an area of interest within the colon;
selecting one set of colonoscopy images for display on a surgical console or highlighting one set of colonoscopy images displayed on a surgical console based on the analyzing; and
switching one autonomous robotic colonoscope cart of the plurality of autonomous robotic colonoscope carts from autonomous control to manual control based on the selecting.

18. The computer-implemented method of claim 17, further comprising:
detecting a collapse in a colon wall based on the received image data; and
generating an alert when the collapse is detected.

19. The computer-implemented method of claim 18, wherein the selecting one set of colonoscopy images for display on a surgical console is based on the analyzing and the detecting the collapse in the colon wall.

20. The computer-implemented method of claim 17, further comprising:
determining whether an image includes surface mucosa; and
causing the autonomous robotic colonoscope cart corresponding to the image to wash the surface mucosa when it is determined that the image includes surface mucosa.

21. A surgical robotic system for conducting an autonomous colonoscopy procedure, the surgical robotic system comprising:
an autonomous robotic colonoscope cart including:
a robotic arm; and
an endoscope operably coupled to the robotic arm and configured to capture images of a colon; and
a surgical console operably coupled to the autonomous robotic colonoscope cart, the surgical console configured to:
receive images captured by the endoscope in real time;
display the received images captured by the endoscope;
receive a destination of a location;
generate a pathway for movement of the endoscope to the received destination;
cause the autonomous robotic colonoscope cart to advance or retract the endoscope through the colon along the generated pathway to the received destination;
analyze the images captured by the endoscope as the endoscope is advanced or retracted through the colon along the generated pathway to the received destination; and
tag an image as corresponding to an area of interest within the colon based on the analysis of the images.

22. The system of claim 21, wherein the surgical console is configured to confirm a location of the endoscope along the pathway based on a comparison between a previously captured image of the colon and a newly captured image of the colon, the newly captured image of the colon captured while the endoscope is advanced or retracted along the generated pathway.

23. The system of claim 21, wherein the destination of the location is based on a selection of a previously captured image.

24. The system of claim 21, wherein the destination of the location is based on a selection of a point along a diagram of the colon.
